SEQUENCE LISTING (18 sequences)

SEQ ID NO: 1 - DNA, 32 bp, Artificial Sequence
  Description of Artificial Sequence Synthetic hsa-miR-17 forward primer
ccctttctcgagccccattagggattatgctg

SEQ ID NO: 2 - DNA, 35 bp, Artificial Sequence
  Description of Artificial Sequence Synthetic hsa-miR-17 reverse primer
gggaaagaattcagatgcaccttagaacaaaaagc

SEQ ID NO: 3 - DNA, 32 bp, Artificial Sequence
  Description of Artificial Sequence Synthetic hsa-miR-601 forward primer
ccctttctcgaggagcacagccctcagtctct

SEQ ID NO: 4 - DNA, 32 bp, Artificial Sequence
  Description of Artificial Sequence Synthetic hsa-miR-601 reverse primer
gggaaagaattctccaactctgactgggacct

SEQ ID NO: 5 - RNA, 21 nt, Artificial Sequence
  Description of Artificial Sequence Synthetic hnRNPA2B1 primer
gcaagaccucauucaauuguu

SEQ ID NO: 6 - RNA, 21 nt, Artificial Sequence
  Description of Artificial Sequence Synthetic hnRNPA2B1 primer
ccauugaaugaggucuugcuu

SEQ ID NO: 7 - RNA, 21 nt, Artificial Sequence
  Description of Artificial Sequence Synthetic siRNA oligonucleotide of hnRNPA2B1
gaacaauggggaaagcuuauu

SEQ ID NO: 8 - RNA, 21 nt, Artificial Sequence
  Description of Artificial Sequence Synthetic siRNA oligonucleotide of hnRNPA2B1
uaagcuuuccccauuguucuu

SEQ ID NO: 9 - RNA, 21 nt, Artificial Sequence
  Description of Artificial Sequence Synthetic siRNA oligonucleotide of nhRNPA2B1
guucagaguucuaggagucuu

SEQ ID NO: 10 - RNA, 21 nt, Artificial Sequence
  Description of Artificial Sequence Synthetic siRNA oligonucleotide of hnRNPA2B1
cacuccuagaacucugaacuu

SEQ ID NO: 11 - RNA, 21 nt, Artificial Sequence
  Description of Artificial Sequence Synthetic siRNA oligonucleotide of hnRNPA2B1
gaagaguaguugagccaaauu

SEQ ID NO: 12 - RNA, 21 nt, Artificial Sequence
  Description of Artificial Sequence Synthetic siRNA oligonucleotide of hnRNPA2B1
uuuggcucaacuacucuucuu

SEQ ID NO: 13 - RNA, 23 nt, Artificial Sequence
  Description of Artificial Sequence Synthetic siRNA control oligonucleotide
aauucuccgaacgugucacguuu

SEQ ID NO: 14 - RNA, 23 nt, Artificial Sequence
  Description of Artificial Sequence Synthetic siRNA control oligonucleotide
acgugacacguucggagaauuuu

SEQ ID NO: 15 - RNA, 23 nt, Unknown
  Description of Unknown Wildtype miR-17 oligonucleotide
caaagugcuuacagugcagguag

SEQ ID NO: 16 - RNA, 23 nt, Unknown
  Description of Unknown Mutant miR-17 oligonucleotide
caaagugcuuacagaggagguag

SEQ ID NO: 17 - RNA, 22 nt, Unknown
  Description of Unknown Wildtype miR-601 oligonucleotide
uggucuaggauuguuggaggag

SEQ ID NO: 18 - RNA, 22 nt, Unknown
  Description of Unknown Mutant miR-601 oligonucleotide
uggucuaggauuguugcacgag

(19) United States
(12) Patent Application Publication
(10) Pub. No.: US 2016/0130577 A1
(43) Pub. Date: May 12, 2016

(54) NUCLEOTIDE SEQUENCE MOTIFS DIRECTING NUCLEIC ACID LOCATION TO EXTRACELLULAR VESICLES

(71) Applicants: FUNDACIÓN CENTRO NACIONAL DE INVESTIGACIONES CARDIOVASCULARES CARLOS III (CNIC), Madrid (ES); UNIVERSIDAD AUTONOMA DE MADRID (UAM), Madrid (ES)

(72) Inventors: Francisco Sánchez Madrid, Madrid (ES); Carolina Villarroya Beltri, Madrid (ES); Maria Mittelbrunn Herrero, Madrid (ES); Cristina Gutiérrez Vázquez, Madrid (ES); Fátima Sánchez Cabo, Madrid (ES)

(21) Appl. No.: 14/431,955

(22) PCT Filed: Sep. 27, 2013
(86) PCT No.: PCT/EP2013/070204

§ 371 Date: Mar. 27, 2015

(30) Foreign Application Priority Data
Sep. 28, 2012 (ES) P 201231503

(51) Int. Cl. C12N 15/113

(52) U.S. Cl. CPC C12N 15/113; C12N 2310/14; C12N 2310/141; A61K 48/00

(57) ABSTRACT

The present invention discloses the use of isolated short sequence motifs capable of directing or packaging regulatory nucleic acids, preferably RNAs, into extracellular vesicles, preferably exosomes. This mechanism is enhanced by the binding of hnRNP family proteins, which are sumoylated, to such nucleic acid. In this sense, sumoylated hnRNPs directs the loading of nucleic acids into EVs through recognition of specific short motifs disclosed in the present invention. Additionally, the present invention discloses recombinant nucleic acids comprising such sequence motifs, EVs in turn comprising these recombinant nucleic acids, as well as the compositions, preferably pharmaceutical compositions comprising either the recombinant nucleic acids or the EVs of the invention. The identification of such motifs is a useful tool for use in genetic engineering and gene therapy.

### FIELD OF THE INVENTION

The present invention can be included in the field of biotechnology and medicine in general, specifically in the field of gene therapy. Particularly, the present invention discloses short sequence motifs capable of directing the nucleic acids which contain them into the extracellular vesicles for excretion from one cell to another in a functionally active form.

### STATE OF THE ART

Extracellular vesicles (EVs) are vesicles having a diameter of 50-300 nm and being excreted and/or secreted by most cells to the extracellular medium either through the fusion of endosomal compartments, called multivesicular bodies, with the plasma membrane, resulting in exosome-type Evs, or via direct release from the plasma membrane resulting in ectosome-type EVs [1].

EVs have a major role in cell-cell communication, having shown that the nucleic acids contained therein, preferably RNA type, including mRNAs, microRNA (miRNA) and other RNAs [2], can be functionally transferred by the secretory or excretory cells and incorporated by the specific recipient cells or target cells where they will perform their function [3-6]. The fact that exosomes contain RNA-type regulatory nucleic acids, suggests their important role in the transfer of genetic information between the different cells in the body. Therefore, the presence of regulatory nucleic acids means that the EVs present potential uses, for example, as biomarkers [7, 8], vaccines [9] and vehicles for gene therapy [10]. Furthermore, by generic engineering methods. EVs can be modified to facilitate and promote the delivery of said nucleic acids or other regulatory molecules within them, in specific target cells [11].

Furthermore, the EVs are not immunogenic and can be captured by the specific recipient cells or target cells and release the functional genetic material within the latter, which would compensate for the function, for example, of defective endogenous genes with no adverse reactions linked to the activation of the immune defences. EVs can also be used in the design of vaccines for immunization against pathogens or tolerability against antigens causing allergies or autoantigens triggering autoimmune diseases.

The existence of specific short nucleotide sequences that present the RNAs and are responsible for directing these RNAs into the nucleus [12] or to the mitochondrion [13] is known in the state of the art.

Despite the growing interest in identifying specific short motifs present in the regulatory nucleic acid sequences and which direct said regulatory nucleic acids into EVs, such motifs are still unknown. The present invention solves said technical problem by finding different specific nucleotide sequence motifs capable of redirecting the regulatory nucleic acids, preferably RNAs into EVs. Identifying said nucleotide sequence motifs is a useful tool for use in genetic engineering, as well as in the redirecting or packaging of specific regulatory nucleic acid into EVs, for therapeutic purposes. Therefore, packaging or redirecting these RNAs into EVs can be useful in designing vaccines to monitor cells which have captured the vesicles (biomarker) or in modulating gene expression in them (gene therapy). Another use may be the modulation of the immune response in a directed and controlled manner in inflammatory, autoimmune processes or even tumours.

### DESCRIPTION OF THE INVENTION

The present invention discloses isolated nucleotide sequence motifs, responsible for redirecting and/or packaging and/or loading nucleic acids, preferably regulatory nucleic acids, preferably RNAs, into EVs.

For purposes of the present invention the term “regulatory nucleic acid” refers to any polynucleotide including a single or double-chain polymer having deoxyribonucleotide or ribonucleotide bases. Nucleic acids may also include modified fragments and nucleotides. In the present invention RNA-type regulatory nucleic acids are preferred and, microRNA (miRNA) type, mRNA type nucleic acids and other RNAs [2] are even more preferred without being limited thereto.

For purposes of the present invention, the term “short sequence motifs” or “short motifs” is also defined as a pattern of specific nucleotide or amino acid sequence presented by the gene or protein sequences, which are associated with a particular function. The reasons described in the present invention are preferably nucleotide motifs and show a sequence length of 2-10 nucleotides, preferably 4-8 nucleotides, and most preferably 4 nucleotides.

For purposes of the present invention, the terms “direct”, “directing”, “redirect” and “redirecting” can be used interchangeably and refer to the action and effect of leading to a specific location. For purposes of the present invention these terms refer to the ability of the specific nucleotide sequence motifs, as defined herein, which have the ability to direct regulatory nucleic acids containing them into EVs.

For purposes of the present invention, the term “package” or “packaging” or “load” or “loading” are used interchangeably throughout this invention, refers to the process of loading or introducing a nucleic acid, protein, molecule or similar into a specific cell organelle. For purposes of the present invention such terms refer to the loading or introduction of regulatory nucleic acid into EVs so that they can be excreted outside the cell.

As used herein, the term “isolated” means separated from the natural state through human intervention. For example, a naturally occurring RNA in a living animal is not “isolated”, but synthetic siRNA, or miRNA or mRNA separated from the coexisting materials of its natural state is considered “isolated”, for purposes of the present invention. Isolated nucleic acid can exist in substantially purified form, or it can exist in a non-native environment such as, for example, a cell into which said nucleic acid has been introduced.

Isolated short nucleotide sequence motifs as described herein and responsible for redirecting and/or packaging the regulatory nucleic acid, preferably RNAs, into the EVs, are those sequences consisting of the nucleotides: GGAG and N₁CCN₂, wherein N₁ is: C or U and N₂ is: U or G. The short nucleotide sequence motifs disclosed in the present invention are preferably located at the 3′region of the regulatory nucleic acid.

The present invention also discloses the association between a protein belonging to the family of heterogeneous nuclear ribonucleoproteins (hnRNPs), specifically the hnRNPA2B1 protein, and regulatory nucleic acid, preferably regulatory RNAs, which present the nucleotide sequence motifs described in the present invention and which are located inside the EVs. Said protein, hnRNPA2B1, along with the nucleotide sequence motifs described in the invention, are responsible of the packaging and/or redirection of regulatory RNAs into the EVs. The protein hnRNPA2B1 specifically binds EVs miRNAs through the recognition of these motifs and controls their loading into EVs, preferably into exosomes. Moreover, hnRNPA2B1 in EVs is sumoylated, and this sumoylation controls the binding of hnRNPA2B1 to miRNAs. The loading of miRNAs into EVs can be modulated by mutagenesis of the identified motifs or changes in hnRNPA2B1 expression levels. The present invention also identifies the hnRNP proteins, preferably hnRNPA2B1, as a key player in regulatory nucleic acids, preferably, miRNA, sorting into EVs.

Suitable heterogeneous nuclear ribonucleoproteins (hnRNPs) useful for the invention include, without limitation: the homo sapiens heterogeneous nuclear ribonucleoproteins A2B1 (hnRNPA2B1) mRNA described in NCBI under accession numbers NM_002137.3 and NM_031243.2 or the polypeptide encoding by that mRNA, described in NCBI under accession numbers NP_002128.1 and NP_112533.1; the homo sapiens heterogeneous nuclear ribonucleoproteins A1 (hnRNPA1) mRNA described in NCBI under accession number NM_002136.2 and NM_031157.2 or the polypeptide encoding by that mRNA, described in NCBI under accession number NP_002127.1 and NP_112420.1. The present invention also contemplates the use of polynucleotides encoding heterogeneous nuclear ribonucleoproteins from different animal species such as, without limitation: Mus musculus, Sus scrofa, Bos Taurus, Xenopus tropicalis, Canis lupus familiaris, Xenopus laevis, Macaca mulatta, Arabidopsis thaliana and Gallus gallus.

So, the first aspect of the present invention refers to the use of the isolated short nucleotide sequence motifs selected from GGAG and N₁CCN₂, or combinations thereof, wherein N₁ is C or U and N₂ is U or G, for the directing and/or packaging of regulatory nucleic acids, preferably RNAs, specifically into EVs. RNAs can be selected from any of those among the list: mRNAs, microRNAs (miRNAs), siRNA and other RNAs.

Moreover, in a preferred embodiment, the isolated short nucleotide sequence motifs disclosed in the invention are used in combination with at least one heterogeneous nuclear ribonucleoprotein (hnRNP) or a modulator thereof. In a more preferred embodiment, the heterogeneous nuclear ribonucleoproteins are also used in combination with at least one modulator of sumoylation.

As used herein, the term “modulator of heterogeneous nuclear ribonucleoprotein” refers to a compound or composition that is capable of the activation or inhibition of the hnRNP activity or expression levels. Modulators of hnRNPs, preferably inhibitors of hnRNPs activity or expression level useful for the invention include, without limitation: chemical compounds, such as RNA aptamer BC15; and biological molecules such as shRNAs/siRNAs for blocking the hnRNPs expression or DNA plasmids for their overexpression.

As used herein the term “sumoylation” refers to the post-translational modifications of cellular proteins by the small ubiquitin-like modifier (SUMO) family of proteins. The sumoylation requires multiple steps that are catalyzed by three types of SUMOylation enzymes: activating enzyme E1 (made up of two subunits, SAE1 and SAE2/Uba2), conjugating enzyme E2 (Ubc9), and one of approximately ten E3 ligases. The sumoylation is reversible by a process known as desumoylation. The removal of SUMO proteins from modified target proteins is accomplished by desumoylation enzymes such as isopeptidase and SUMO/sentrin-specific protease (SENP).

As used herein, the term “modulator of sumoylation” refers to a compound or composition that is capable of affecting directly (activation) or indirectly (inhibition) the sumoylation of a protein. In some embodiments, the modulator of sumoylation will negatively control sumoylation i.e. directly or indirectly prevent sumoylation and/or reverse sumoylation by a desumoylation process. In yet other embodiments, the modulator of sumoylation will positively control sumoylation i.e. directly or indirectly bring about sumoylation. Modulators of sumoylation, preferably inhibitors of sumoylation useful for the invention include, without limitation: ginkolic acid and anacardic acid, which are inhibitors of the Ubc9 enzyme. In another preferred embodiment of the invention, modulators of sumoylation, preferably inhibitors of sumoylation useful for the invention include, without limitation the latent membrane protein 1 (LMP-1) from the Epstein-Barr virus.

Another aspect of the present invention relates to the use of specific short nucleotide motifs selected from AN₃CAUN₁, AGGUAGUA and N₁UGCACUN₄ or combinations thereof, wherein N₁: C or U; N₂: U or G; N₃ C or A and N₄: G or A, to retain regulatory nucleic acid, preferably RNAs, within the cell. RNAs can be selected from any of those among the list: mRNAs, microRNAs (miRNAs), siRNA and other RNAs.

For purposes of the present invention, the term recombinant nucleic acid refers to any nucleic acid molecule, DNA or RNA, obtained artificially by molecular biology techniques, either by binding or interleaving DNA or RNA sequences into a recipient DNA or RNA, other than the donor nucleic acid, or by directed mutagenesis techniques, altering the specific sequence of a nucleic acid by this technique or any other technique known and used by anyone skilled in the art.

Another aspect of the present invention relates to isolated nucleic acids themselves, preferably recombinant nucleic acid, preferably RNAs comprising at least one short motif selected from GGAG and N₁CCN₂, or combinations thereof, wherein N₁: C or U and N₂: U or G and that are capable of being loaded or introduced into EVs.

Another aspect of the present invention relates to isolated nucleic acids, preferably recombinant nucleic acid, preferably RNAs comprising at least one specific short motif selected from AN₃CAUN₁, AGGUAGUA and N₁UGCACUN₄ or combinations thereof, wherein N₁: C or U; N₂: U or G; N₃ C or A and N₄: G or A, which are retained within the cell and are excreted outside the cell.

Another aspect of the present invention relates to EVs comprising at least one recombinant nucleic acid, preferably RNA, comprising at least one short sequence motif selected from GGAG and N₁CCN₂, or combinations thereof, wherein N₁: C or U and N₂: U or G, capable of being loaded or introduced into EVs, as previously described herein. In a preferred embodiment, the short nucleic motifs present in the regulatory nucleic acids contained in the EVs are in combination with at least one heterogeneous nuclear ribonucleoprotein (hnRNP) or a modulator thereof. In a more preferred embodiment, the heterogeneous nuclear ribonucleoproteins mentioned are in combination with at least one modulator of sumoylation.

The EV motifs can be used in genetic engineering for redirecting or packaging specific regulatory nucleic acid into EVs. These EVs can be used as devices for gene therapy, since they are non-immunogenic extracellular vesicles that can be taken up by specific target cells and release functional genetic material into them. This would allow to silence or compensate the function of defective genes in the recipient cells.

EVs can be used as vaccines for the immunization against pathogens or the tolerization against allergens or autoantigens in autoimmune diseases [1,9]. The selective packaging of regulatory nucleic acids, preferably, RNAs, into these vesicles can be used in the design of vaccines.

Moreover, it is important to note that EVs are secreted by many immune cells and tumour cells, and they play a key role in the modulation of immune responses against pathogens and tumours [1]. The packaging of regulatory RNAs, such as miRNAs in these vesicles can be useful for control and modulate the immune response in inflammatory diseases, autoimmune diseases or tumours.

Another aspect of the present invention relates to a composition comprising at least one EV as previously defined, and further, at least one pharmaceutically acceptable carrier or excipient. Said composition is preferably a pharmaceutical composition and may be selected from any of the following: a gene therapy composition, a vaccine or a biomarker, among others.

The pharmaceutical composition of the invention, if desired, may also contain, when necessary, additives to increase, control or otherwise direct the desired therapeutic effect of regulatory nucleic acids having the nucleotide sequence motifs of the invention and EVs containing such nucleic acids. Such additives and/or auxiliary substances or pharmaceutically acceptable substances, either excipient or carrier type, can be selected from any of the following: buffering agents, surfactants, cosolvents, preservatives, disintegrants, diluents, etc. Said pharmaceutically acceptable substances which can be used in the pharmaceutical composition of the invention are known generally to those skilled in the art. Examples of suitable pharmaceutical carriers are described, for example, in “Remington's Pharmaceutical Sciences” by E. W. Martin. Additional information is available on such vehicles in any pharmaceutical technology manual (Galenic Pharmacy).

The term “pharmaceutically acceptable carrier or excipient” refers to a vehicle that must be approved by a regulatory agency of the Federal or a state government or listed in the U.S. Pharmacopoeia or the European Pharmacopoeia or other generally recognized pharmacopeia for use in animals, and particularly in humans.

For purposes of the present invention the term “biomarker” refers to any substance used as an indicator of a biological state or of a normal biological process, a disease state or a response to a drug treatment. For purposes of the present invention, the term biomarker refers to regulatory nucleic acids, preferably RNAs and more preferably, mRNAs, miRNAs, siRNAs, or other RNAs.

For purposes of the present invention the term “vaccine” refers to a preparation or composition comprising the EVs of the invention or alternatively the isolated nucleic acids of the invention, which once inside the body cause the production of antibodies and thus a defence response against pathogens. The vaccines described herein can be used against pathogenic organisms or to induce tolerance against antigens which cause allergies or against autoantigens triggering autoimmune diseases.

For purposes of the present invention the term “gene therapy” refers to any process capable of inserting functional genetic material into a cell, to correct a genetic and/or metabolic, physiological and/or functional defect, either by over-expression or under-expression, or to provide the cells with a new function. Specifically, the term gene therapy, for the purpose of the present invention concerns the use of EVs or of the isolated nucleic acids of the invention for the treatment of diseases, to restore or inhibit genes, proteins or specific functions in the cell.

For purposes of the present invention, the term “effective dose” refers to the minimum dose capable of producing the desired effect, whether the reversion of a disease state, or inducing a specific immune response, etc.

The term “expression” as used herein, refers to the transcription of a gene sequence as well as the translation of an mRNA to the resulting protein. As used herein, the term “gene” refers to nucleic acid (e.g. DNA or RNA) comprising the partial or full-length sequence coding the sequences necessary for the production of a polypeptide or a polypeptide precursor.

As used herein, the term “silencing” refers to the suppression or inhibition of the expression of a specific target gene. The term silencing does not necessarily imply reduced transcription as the gene silencing also, at least in some instances, may be performed at the post-transcriptional level. The degree of gene silencing can occur to suppress or interfere with the production of the encoded gene product.

Another objects described in the present invention refer to the use of isolated nucleic acids themselves, or use of EVs themselves or use of the compositions described herein, in the manufacture of a medicine, preferably a medicine for gene therapy, vaccine or a biomarker.

Another objects described in the present invention relate to isolated nucleic acids themselves, or the EVs themselves or the compositions described herein, for use as medicines, preferably for gene therapy, as vaccines or as biomarkers.

Another objects described in the present invention relates to a method of treating diseases by administering to a subject an effective dose of the isolated nucleic acids or EVs or of the compositions described in this invention.

Another objects described in the present invention refer to a method of treatment of immunological and inflammatory diseases and tumour processes, by administering to a subject an effective dose of the isolated nucleic acids or EVs or of the compositions described herein.

Another of the objects described in the present invention refer to a method of vaccination by administering to a subject at least one effective dose of a vaccine comprising the isolated nucleic acids or EVs or compositions, as described herein.

Unless otherwise defined, all technical and scientific terms used herein have the meaning commonly understood by one skilled in the art to which this invention belongs. The following references provide one of ordinary skill in the art with a general definition of many of the terms used in this invention: Singleton et al. Dictionary of Microbiology and Molecular Biology (3rd edition 2006.) Cambridge Dictionary of Science and Technology (Walker ed., 1990) Glossary of Genetics, 5th Ed, R. Rieger et al. (eds.), Springer Verlag (1991) and Hale & Marham, The Harper Collins Dictionary of Biology (1991). As used herein, all terms have the meanings ascribed to them and known in the prior art unless otherwise specified.

### DESCRIPTION OF THE FIGURES

FIG. 1 (A) Microarray heat map showing the expression of different miRNAs in cells (CL) and in EV in activation (ACT) or resting (REST) condition. Darker shades relate to increased expression and lighter shades to lower expression. (B) The upper panel shows the Venn diagrams obtained based on the miRNA microarray results. UP: over-expression, Down: under-expression, CL: Cell, EV: extracellular vesicle; ACT: activation and REST: rest. (C) Venn diagrams obtained from the mRNA microarray results. UP: over-expression, Down: under-expression, CL: Cell, EV: extracellular vesicle; ACT: activation and REST: rest.

FIG. 2. Interaction between miRNAs and their mRNAs inside the EVs (white bars) or within cells (black bars), both in rest (A) and activation (B) conditions. MiEV: EVmiRNA; MeV: EVmRNA; miCL: CLmiRNA; mCL: CLmRNA. The Y axis shows the number of interactions between miRNAs and mRNAs in EVs and in cells.

FIG. 3. Multiple alignment cladogram of the sequences of mature miRNAs. The miRNAs in a darker shades are located in cells (dark gray) and those in a lighter shade are located in EVs (light gray).

FIG. 4. Wild-type (Wt) and mutated (Mut) sequences of the miRNAs miR-17 (A) and miR-601 (B), showing the CLmotifs and EVmotifs.

FIG. 5. The upper panel shows the graphs representing the levels of expression (qPCR) of miR-17 wt and mutated (17mut) in control cells (non-transfected), in cells transfected with miR-17 wt and in cells transfected with miR-17mut. The lower panel shows the graphs representing levels of expression (qPCR) of miR-601 wt and mutated (601mut) in control cells, in cells transfected with miR-601 wt and in cells transfected with miR-601mut. The Y-axis shows expression levels expressed in arbitrary units (AU).

FIG. 6. Graphs obtained by flow cytometry analysis showing the expression of the fluorescent protein GFP in control cells (non-transfected) (A) and in cells transfected with plasmids expressing miRNAs miR-17 wt (B), miR-17-mut (C), miR-601 wt (D) or miR-601 mut (E).

FIG. 7. (A) Graph showing the ratio EV/CL of the miRNAs miR-18A, miR-17, miR-198 and miR-601. Error bars represent standard deviation (n=3). (B) Graph showing the ratio EV/CL of miRNA-17 wild-type (wt) and mutated (mut) and of miR-18a wild-type (wt). Error bars represent standard deviation (n=3). *P<0.05. (C) Graph showing the ratio EV/CL of miRNA-601 wild-type (wt) and mutated (mut) miR-18a wild-type (wt). Error bars represent standard deviation (n=3). *P<0.05.

FIG. 8. (A) Photographs of Western Blot which shows the presence of different proteins from the hnRNPs protein family in cells (CL) and their EVs. The numbers represent the molecular weight of each of the proteins expressed in kDa. (B) Photograph of Western Blot showing immunoprecipitation of the hnRNPA2B1 protein. CH: heavy chain of the antibody used in the immunoprecipitation. CL: light chain of the antibody used in the immunoprecipitation. Anti-hnRNPA2B1: immunoprecipitation with an Anti-hnRNPA2B1. Anti-IgG1: immunoprecipitation with an IgG1 isotype control antibody. No Ab: no antibody immunoprecipitation. (C) FACS analysis of hnRNPA2B1 and CD81 in EV-coupled beads. EVs were coupled to aldehyde-sulfate beads, permeabilized or left intact, and incubated with antibodies to hnRNPA2B1 (middle panels) or CD81 (right panels) and secondary antibody. EV-coupled beads incubated with secondary antibody alone were used as negative controls.

FIG. 9. (A) A graph showing gene expression (qPCR, in arbitrary units) of the miR-17 (white bars) and miR-198 (black bars) in immunoprecipitation assays of hnRNPA2B1 protein. EVs were incubated with magnetic beads coated with anti-IgG1 (control) or with anti-hnRNPA2B1. Error bars represent standard deviation (n=2). (B) Electrophoretic mobility shift assay (EMSA) showing the specific binding of miR-198 to hnRNPA2B1 protein. The signs (+) indicate presence and signs (−) absence. (C) Electrophoretic mobility shift assay showing the binding of hnRNPA2B1 to wild-type and mutated miR-198. Biotinylated wild-type and mutated miR-198 were incubated with or without purified human hnRNPA2B1 as indicated. Numbers represent protein concentration (ng/μl). (D) Electrophoretic mobility shift assay showing the binding of hnRNPA2B1 to miR-601.

FIG. 10. (A) Photograph of Western blot which shows hnRNPA2B1 silencing. The numbers represent the molecular weight of each of the proteins expressed in kDa. (B) Graphic of flow cytometry (FACS) showing over-expression of the protein hnRNPA2B1-GFP.

FIG. 11. (A) Graphic showing the expression (qPCR) of miRNAs miR-18a and miR-198 in EVs of control cells (white bars) or of cells not expressing the protein hnRNPA2B1 in being transfected with a specific siRNAs against the same (black bars). Error bars represent s.e.m. (n=3). p<0.001. (B) Graph of the expression (qPCR) of the miRNAs miR-18a and miR-198 in EVs of control cells (white bars) or of cells overexpressing the hnRNPA2B1 protein (black bars). Error bars represent s.e.m.

FIG. 12. hnRNPA2B1 is sumoylated in EVs and this modification controls its binding to miRNAs. (A) Representative western blot analysis showing hnRNPA2B1 in T cells and their EVs. (B) Western blot analysis of hnRNPA2B1 sumoylation. HEK293T cells were co-transfected with SUMO-1 and hnRNPA2B1-GFP or GFP plasmids. GFP immunoprecipitates and total lysates were immunoblotted for SUMO-1 and/or GFP. Ab: Antibody-conjugated Dynabeads without cell lysates. GFP: Lysates from cells transfected with GFP and SUMO-1. hnRNPA2B1-GFP: Lysates from cells transfected with hnRNPA2B1-GFP and SUMO-1. (C) Western blot analysis of hnRNPA2B1 sumoylation in T cells. hnRNPA2B1 was immunoprecipitated from Jurkat T cells and immunoblotted for SUMO-1 and hnRNPA2B1. A2B1: hnRNPA2B1; s-A2B1: sumoylated hnRNPA2B1; ns: non-specific band. IP CONT: immunoprecipitation with control antibody. IP A2B1: immunoprecipitation with hnRNPA2B1 antibody. (D) Western blot analysis of hnRNPA2B1 molecular weight in EVs and cells in the presence of the sumoylation inhibitor anacardic acid (AA) or vehicle (DMSO). Numbers below the lanes are the densitometry ratios of total hnRNPA2B1 to sumoylated hnRNPA2B1. (E) qPCR analysis of miRNA levels in EVs from control or AA-treated cells. Bars represent miR-17, miR-18 and miR-198 levels (arbitrary units). Error bars represent standard deviation (n=3). Students's t test; *p-value<0.05. (F) qPCR analysis of miR-198 in hnRNPA2B1 immunoprecipitates from EV lysates derived from control or AA-treated cells, showing decreased binding of miR-198 to hnRNPA2B1 in the presence of AA. Bars represent miR-198 levels (arbitrary units). Data are presented relative to the control condition. Error bars represent standard deviation (n=3). Student's t-test; *p-value<0.05.

FIG. 13. Assessment of sumoylation inhibitor efficiency. (A) Western blot analysis of hnRNPA1 in T cells and their EVs. (B) Representative western blot showing the decrease in sumo-conjugated proteins in the presence of anacardic acid. Cells were incubated with anacardic acid (AA) or DMSO (control condition, CONT) and cell extracts were blotted for SUMO-1. (C) Representative western blot showing no changes in EV secretion and cell death in the presence of anacardic acid. Cells were incubated with anacardic acid (AA) or DMSO (CONT), and EV were purified by ultracentrifugation and blotted for CD81 and cytochrome c.

### DETAILED DESCRIPTION OF THE INVENTION

The first object of the present invention relates to the extracellular vesicles (EVs) comprising at least one isolated regulatory nucleic acids having at least one short nucleic motifs selected from GGAG and N₁CCN₂, wherein N₁: is C or U and N₂ is U or G; or combinations thereof. In a preferred embodiment of the invention, the short nucleic motifs are located at the 3′region of the regulatory nucleic acid.

In a preferred embodiment of the invention, the short nucleic motifs present in the regulatory nucleic acids of the extracellular vesicles are in combination with at least one heterogeneous nuclear ribonucleoprotein (hnRNP), or a modulator thereof. In a more preferred embodiment of the invention, the heterogeneous nuclear ribonucleoproteins are in combination with at least one modulator of sumoylation. In another preferred embodiment of the invention, the heterogeneous nuclear ribonucleoprotein is selected from: hnRNPA2B1 and hnRNPA1. Preferably, the modulators of heterogeneous nuclear ribonucleoproteins are selected from inhibitors or activators thereof. More preferably, the inhibitors of heterogeneous nuclear ribonucleoproteins are selected from: RNA aptamers, such as BC15. More preferably, the activators of heterogeneous nuclear ribonucleoproteins are selected from: specific plasmid for their overexpression.

In another preferred embodiment the modulator of sumoylation are selected from inhibitors or activators thereof. Preferably, the inhibitors of sumoylation are selected from: inhibitors of Ubc9 activity, preferably, ginkolic acid or anacardic acid. In another preferred embodiment of the invention, modulators of sumoylation, preferably inhibitors of sumoylation useful for the invention include, without limitation the latent membrane protein 1 (LMP-1) from the Epstein-Barr virus.

In another preferred embodiment of the invention, the regulatory nucleic acid included in the EVs is preferably, DNAs and RNAs, more preferably, RNAs and most preferably selecting from: mRNA, miRNA, siRNA and other RNAs.

In a more preferred embodiment, the EVs of the invention are preferably exosomes.

Another object of the present invention relates to isolated regulatory nucleic acids characterized in that they comprise at least one short motif selected from between GGAG and N₁CCN₂, wherein N₁ is C or U and N₂ is U or G; or combinations thereof.

In a preferred embodiment, the isolated regulatory nucleic acids are in combination with at least one heterogeneous nuclear ribonucleoprotein (hnRNP), or a modulator thereof. In a more preferred embodiment of the invention, the heterogeneous nuclear ribonucleoproteins are in combination with at least one modulator of sumoylation. In another preferred embodiment of the invention, the heterogeneous nuclear ribonucleoprotein is selected from: hnRNPA2B1 and hnRNPA1. Preferably, the modulators of heterogeneous nuclear ribonucleoproteins are selected from inhibitors or activators thereof. More preferably, the inhibitors of heterogeneous nuclear ribonucleoproteins are selected from: RNA aptamers such as BC15. More preferably, the activators of heterogeneous nuclear ribonucleoproteins are selected from: specific plasmid for their overexpression.

In another preferred embodiment the modulator of sumoylation are selected from inhibitors or activators thereof. Preferably, the inhibitors of sumoylation are selected from: inhibitors of Ubc9 activity, preferably, ginkolic acid or anacardic acid. In another preferred embodiment of the invention, modulators of sumoylation, preferably inhibitors of sumoylation useful for the invention include, without limitation the latent membrane protein 1 (LMP-1) from the Epstein-Barr virus.

In a preferred embodiment of the invention, the isolated regulatory nucleic acids are preferably DNAs and RNAs, and more preferably, RNAs. In a most preferred embodiment, the isolated regulatory nucleic acids are selected from any of the following list: mRNA, miRNA, siRNA and other RNAs.

In another preferred embodiment, the isolated nucleic acids of the invention are characterized in that they are recombinant regulatory nucleic acids.

In another preferred embodiment, the isolated nucleic acids of the invention are characterized in that they can be introduced into the interior of the EVs.

Another object of the present invention relates to a composition comprising at least one extracellular vesicle as defined in the present invention or at least one isolated regulatory nucleic acid as defined in the present invention. In a preferred embodiment, the composition of the invention is a pharmaceutical composition.

In a preferred embodiment, the composition and preferably, the pharmaceutical composition of the invention further comprises at least one pharmaceutically acceptable carrier or excipient. In a more preferred embodiment, the pharmaceutical composition is selected from: a composition for gene therapy, a vaccine or a biomarker.

Another object of the present invention relates to the use of isolated short nucleotide motifs selected from GGAG and N₁CCN₂, wherein N₁ is C or U and N₂ is U or G; or combinations thereof, for loading regulatory nucleic acids into extracellular vesicles. In a preferred embodiment, the use of the short nucleotide motifs of the invention is in combination with at least one heterogeneous nuclear ribonucleoprotein (hnRNP), or a modulator thereof. In a more preferred embodiment of the invention, the heterogeneous nuclear ribonucleoproteins are in combination with at least one modulator of sumoylation. In another preferred embodiment of the invention, the heterogeneous nuclear ribonucleoprotein is selected from: hnRNPA2B1 and hnRNPA1. Preferably, the modulators of heterogeneous nuclear ribonucleoproteins are selected from inhibitors or activators thereof. More preferably, the inhibitors of heterogeneous nuclear ribonucleoproteins are selected from: RNA aptamers such as BC15. More preferably, the activators of heterogeneous nuclear ribonucleoproteins are selected from: specific plasmid for their overexpression.

In another preferred embodiment the modulator of sumoylation are selected from inhibitors or activators thereof. Preferably, the inhibitors of sumoylation are selected from: inhibitors of Ubc9 activity, preferably, ginkolic acid or anacardic acid. In another preferred embodiment of the invention, modulators of sumoylation, preferably inhibitors of sumoylation useful for the invention include, without limitation the latent membrane protein 1 (LMP-1) from the Epstein-Barr virus.

In another preferred embodiment, the use of isolated short nucleotide motifs is characterized in that the regulatory nucleic comprising such short nucleotide motifs are preferably, DNAs and RNAs, more preferably, RNAs and most preferably selecting from: mRNA, miRNA, siRNA and other RNAs.

In a more preferred embodiment, the use of isolated short nucleotide motifs according to the present invention is characterized in that the EVs are preferably exosomes.

Another aspect of the present invention relates to the use of isolated short nucleotide motifs selected from AN₃CAUN₁, AGGUAGUA and N₁UGCACUN₄ or combinations thereof, wherein N₁: C or U; N₂: U or G; N₃: C or A and N₄: G or A, to retain nucleic acids within the cell. The nucleic acids are preferably regulatory nucleic acids, preferably DNAs or RNAs, and more preferably RNAs, and most preferably those selected from any of the list: mRNAs, microRNA (miRNA), siRNA and other RNAs.

Another aspect of the present invention relates to isolated nucleic acids, characterized in that they comprise at least one short motif selected from AN₃CAUN₁, AGGUAGUA and N₁UGCACUN₄ or combinations thereof, wherein N₁: C or U; N₂: U or G; N₃ C or A and N₄: G or A, capable of being sequestered inside the cell.

In a preferred embodiment, such isolated nucleic acids are characterized in that they are regulatory nucleic acids, preferably DNA and RNA, and more preferably RNA.

In another preferred embodiment, the regulatory isolated nucleic acids are selected from any of the following list: mRNA, miRNA, siRNA and other RNAs.

In another preferred embodiment, the isolated nucleic acids of the invention are characterized in that they are recombinant regulatory nucleic acids.

In another preferred embodiment, the isolated nucleic acids of the invention are characterized in that they can be retained inside the cell and are not excreted outside the cell.

Another object of the present invention relates to the use of the isolated nucleic acids, or to the use of EVs, or to the use of the composition described herein, in the manufacture of a medicament, said medicament preferably being a gene therapy medicament. In a preferred embodiment, the isolated nucleic acids or the EVs themselves or the compositions described in the present invention are used for the manufacture of a medicament for the treatment of immune diseases, inflammatory diseases and tumour processes.

Another object described in the present invention refers to the use of the isolated nucleic acids, or the use of the EVs, or the use of the composition described herein for the preparation of biomarkers or for the preparation of vaccines.

Another object of the present invention relates to the isolated nucleic acids or the EVs, or the compositions described herein for use as medicaments, preferably a medicament for use in gene therapy.

In another object of the invention, the isolated nucleic acids or the EVs, or the compositions described herein are used for the treatment of immune diseases, inflammatory diseases and tumour processes.

Another object of the present invention relates to isolated nucleic acids or EVs, or compositions described herein, for use as biomarkers or for use as vaccines.

Another object of the present invention refers to a method for the treatment of immune diseases, inflammatory diseases and tumour processes, characterized in that comprising the administration to a subject in need thereof at least one effective dose of the isolated nucleic acids or of the EVs or of the compositions of the invention.

Another object described in the present invention relates to a method of treating diseases, preferably by gene therapy, comprising the administration to a subject in need thereof at least one effective dose of the isolated nucleic acids or of the EVs or of the compositions of the invention.

Another object of the present invention relates to a method of vaccination which comprises administering to a subject in need thereof, of at least one effective dose of a vaccine comprising the isolated nucleic acids, or the EVs themselves, or the compositions of the invention.

The examples listed below are intended to illustrate the invention without limiting the scope thereof.

### EXAMPLES

### Example 1

### Study of Differential Presence of miRNAs and mRNAs in Cells and in EVs

To obtain the results shown in the present invention, human mononuclear cells from peripheral blood, isolated from healthy donors and separated in Ficoll (Biochrom) were used. To enhance their adherence, they were maintained for 30 min at 37° C. After this time, the non-adherent cells were collected and cultured for 2 days in the presence of phytohemagglutinin (5 mg/ml) to induce lymphocyte proliferation. To obtain T lymphoblasts, a concentration of 50 U/ml of interleukin-2 (IL-2) was progressively added to the culture medium every 2 days over a period of 8 days. In order to obtain T lymphoblasts in activation state, 50 mg/ml of phorbol myristate acetate (PMA) and ionomycin (500 ng/ml) were added to the culture medium.

In addition to human T lymphoblasts, the present invention also used the human cell line derived from T cells, called Jurkat J77c120 (TCR Val Vβ8) which was cultured in RPMI medium (Sigma), to which 10% foetal bovine serum (FBS, Invitrogen) was added.

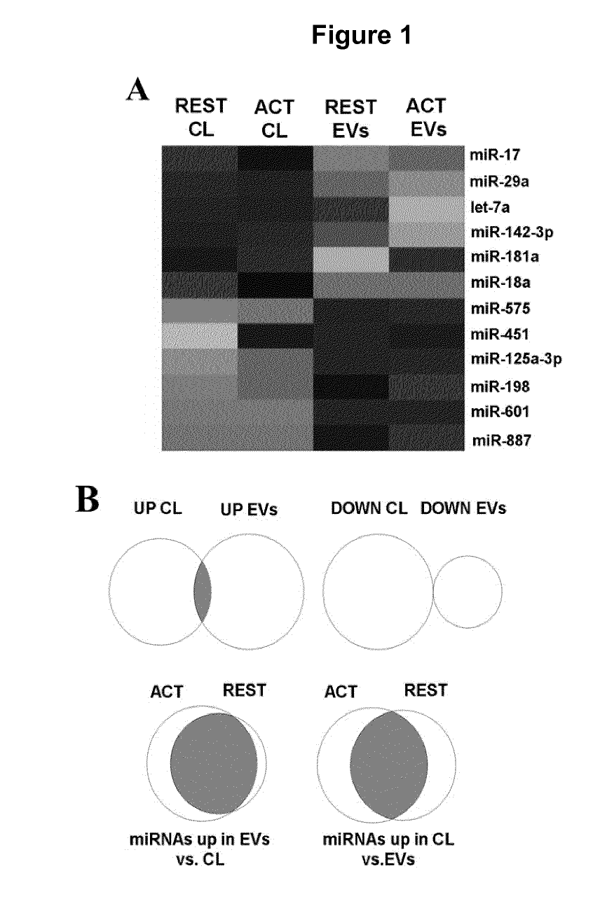

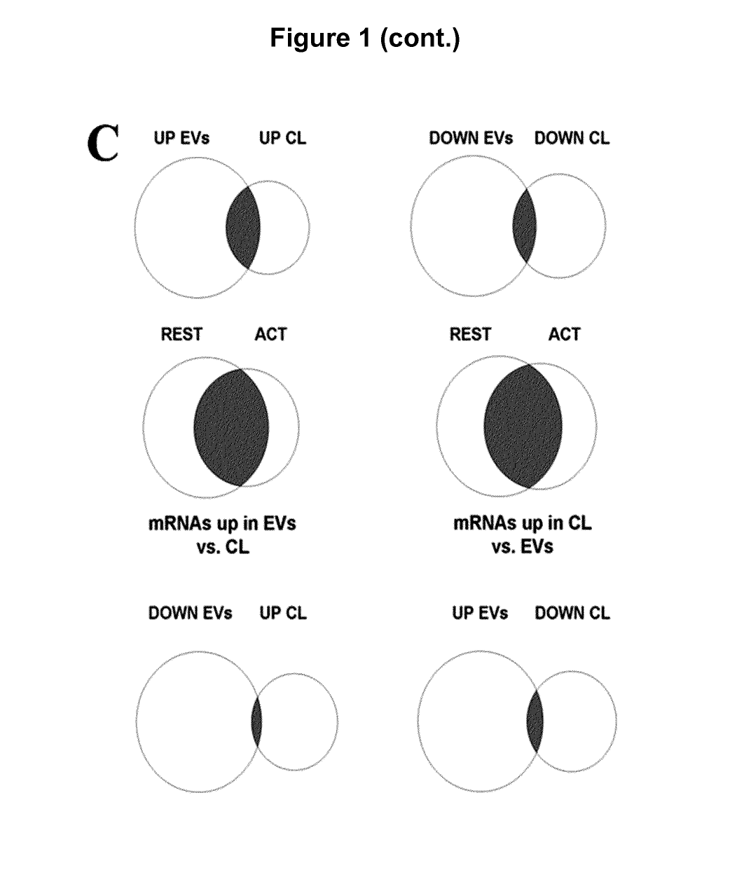

To analyze the existence of specific miRNAs that are specifically located inside the EVs compared to those miRNAs that are specifically localized in the cell cytoplasm, the microarray technique was used (FIG. 1A). This technique makes it possible to obtain an analysis of the expression of the changes induced in the expression profiles of miRNAs and mRNAs, both in cells, activated and resting primary T lymphoblasts, and in EVs isolated from these activated and resting T lymphoblasts.

Vesicle isolation from activated or resting T lymphoblasts was carried out using cultured primary human T lymphoblasts obtained from eight donors and EVs isolated from such lymphoblasts, in RPMI-1640 medium supplemented with FBS 10%, as indicated above, both at rest and after activation with PMA (50 ng/ml) and ionomycin (500 ng/ml). To ensure that the culture medium was free of EVs, it was centrifuged at 100,000 g overnight. Activation of the cells was assessed by testing the over-expression of CD69 protein by flow cytometry on a FACSCanto cytometer (BD Biosciences) and using FACSDiva software (BD Biosciences). EVs were obtained from the cell supernatants by different centrifugation and filtering stages [3]. Briefly cells were centrifuged (320 g for 5 min) and the supernatant was filtered through membranes with 0.22-μm pores. EVs were pelleted by ultracentrifugation at 100,000 g for 60 min at 4° C. (Beckman Coulter Optima L-100 XP, Beckman Coulter), thus providing the EVs isolated from resting and activated T lymphoblasts. Moreover, when it is indicated, the EVs were overlaid with a linear sucrose gradient (2.5-0.4M sucrose in PBS) and floated into the gradient by centrifugation for 16 h at 120,000 g. For the analysis of miRNA profile in cells overexpressing hnRNPA2B1. EVs were isolated with ExoQuick-TC Exosome Precipitation Solution (SBI).

After obtaining the cells and EVs, total RNA isolation was performed using QIAzol reagent (Qiagen) and miRNeasy mini kit (Qiagen). The purity and concentration was then analyzed in the Nanodrop-1000 spectrophotometer (Thermo Scientific), as well as its integrity by ethidium bromide staining on agarose gel 1.5%. For the analysis of the profiles of the long and short RNAs present in the cells and in the EVs. Agilent 2100 Bioanalyzer (Agilent) was used both for the total RNA (RNA nanochips) and for short RNAs (short RNA chips). Microarray assays were performed using Agilent human miRNA and mRNA microarrays. Each array was used to analyze two groups of RNAs obtained from primary human T lymphoblasts and their purified EVs, both resting and activated, isolated and cultured from samples of four healthy donors. The data obtained for each of the arrays, miRNA and mRNA were normalized using the quartiles method (similar results were obtained for miRNA when results were normalized using the vsn2method). After normalization, only those probes of miRNA present in at least two samples and with an average of expression greater than 20 percentile of the total average of expression (316 remaining miRNAs) were considered for subsequent analysis. Similarly, the probes of genes included in the assay of mRNA were excluded from analysis if they failed to show a strong signal and an acceptable indicator in all replicates of at least one condition studied. Additionally they were asked to present any changes through the samples (CV>5%). Linear models [14] as implemented in the Bioconductor lima package were used to find differentially expressed miRNAs and mRNAs between cells and EVs. A differential expression was deemed to exist both in genes (mRNAs) and miRNAs when ADJP≦0.05.

FIG. 1 shows the results of the microarray. These results demonstrate that most of the miRNAs that are modulated (either by an increase or decrease in their expression) under activation conditions are not the same in the cells as in the EVs (FIG. 1B, upper panel). Thus, miRNAs that increase their expression levels in activated T lymphoblasts, do not show, however, such significant increase in EVs. A similar trend was observed for the mRNA (FIG. 1C), i.e. most of the mRNA which are modulated (either by increased or decreased expression) at rest or activation, are different in the cells compared to EVs.

A mismatch between the profiles of the miRNA and mRNAs in activation conditions between cells and EVs demonstrates that miRNAs and mRNAs are not directed into the EVs in a random or passive manner.

Moreover, as can be seen in FIG. 1A, various miRNAs were represented to a greater extent in EVs than in cells, such as the miRNAs: miR-575, miR-451, miR-125a-3p, miR-198, miR-601 and miR-887. It is also noteworthy that, in most cases, this difference is maintained under both cellular rest and activation conditions (FIG. 1B, lower left panel and FIG. 1C). In turn, most of the miRNAs that show a greater representation in the cells with regard to EVs, such as miRNAs: miR-17, miR-29a, let-7a, miR-142-3p, miR-181a and miR-18a (FIG. 1A), maintain such trends regardless of the state of cell activation (FIG. 1B, lower right panel and FIG. 1C central right panel). These data indicate that there are miRNAs and mRNAs located specifically inside the EVs irrespective of the activation state of the cell, whereas there are other miRNAs and mRNAs which are located specifically within the cell.

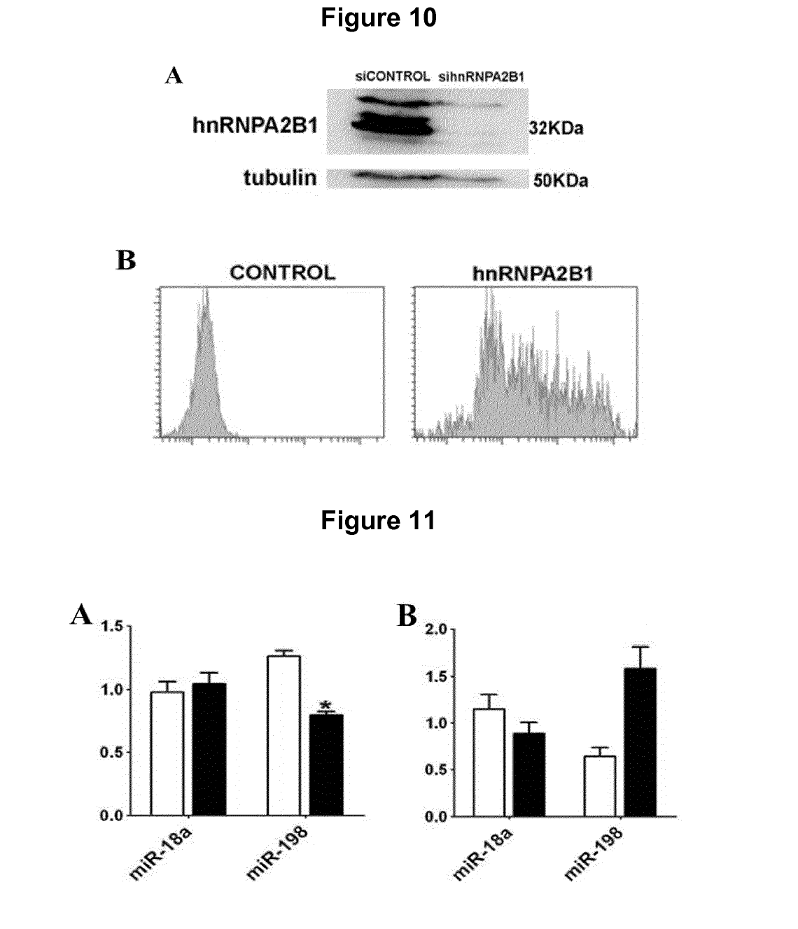

Throughout the present invention, the miRNAs or mRNAs that are specifically located inside the EVs will be referred to as EVmiRNAs or EVmRNAs respectively, while the miRNAs or mRNAs that are located inside the cell will be referred to as CLmiRNAs or CLmRNAs respectively. Similar results, as seen in FIG. 10, were obtained when the expression analysis was performed using microarray for the case of mRNAs (FIG. 10).

Therefore, the data shown herein demonstrate the existence of specific mechanisms that control the active directing or steering of RNAs, either miRNAs, mRNAs, or any other type of regulatory nucleic acid into the EVs. Similarly, it also reveals the existence of specific mechanisms that cause RNAs to remain retained inside the cell and to not be secreted to the outside.

To check whether miRNAs were packaged in EVs together with their target mRNAs, an analysis was performed on the interaction of mRNAs and miRNAs present in the EVs and those present within the cell. The number of possible interactions between the EVmiRNAs and EVmRNAs and between the EVmiRNAs and CLmRNAs was measured, both at rest and activation, respectively, using databases of miRNA-target mRNA interaction both computationally predicted and experimentally validated.

To carry out this analysis, miRNAs and mRNAs taken were those that showed a statistically significant (p≦0.05) positive (upward) or negative (downward) regulation, and differentially represented in EVs compared to cells. The target miRNA were obtained from four databases containing validated experimental interactions: Tarbase, miRTarBase, miRWalk and miRecords. MiRNA-mRNA interactions were obtained from nine sources: EIMMo, DIANA-microT, Microcosm, Microrna.org, TargetScan, Mirtarget, PITA, miRWalk-predictive and TargetSpy. The interactions that showed no significant mRNAs or miRNAs were not considered. The scores for each predictive algorithm were normalized and the combination of predictive and experimental databases was calculated [15]. The resulting miRNA-gene pairs were then grouped according to their level of expression and resting or activation conditions.

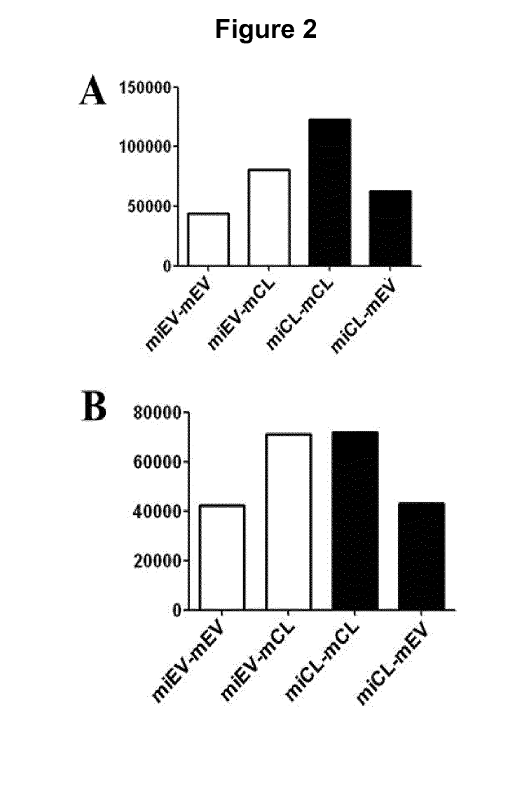

The results obtained by the miRNA-mRNA interaction tests revealed that EVmiRNAs and CLmRNAs have a higher level of interaction, approximately twice as much, both at rest (FIG. 2A) and activation (FIG. 2B), as those showed by the EVmiRNAs and EVmRNAs. These results show, in general, that the miRNAs and their target mRNAs are not packaged together in EVs.

### Example 2

### Analysis of the Sequence Motifs Responsible for the Packaging of the RNAs within the EVs

To analyze the differences in the sequences of the EVmiRNAs and CLmiRNAs, a multiple alignment analysis (ClustalW, gap open 12, gap extension 3) was performed on the mature miRNA sequences that showed differential expression, both at rest and in activation conditions, in cells with regard to EVs. Such analysis was performed on human primary T lymphoblasts and on the cell line of Jurkat T lymphocytes.

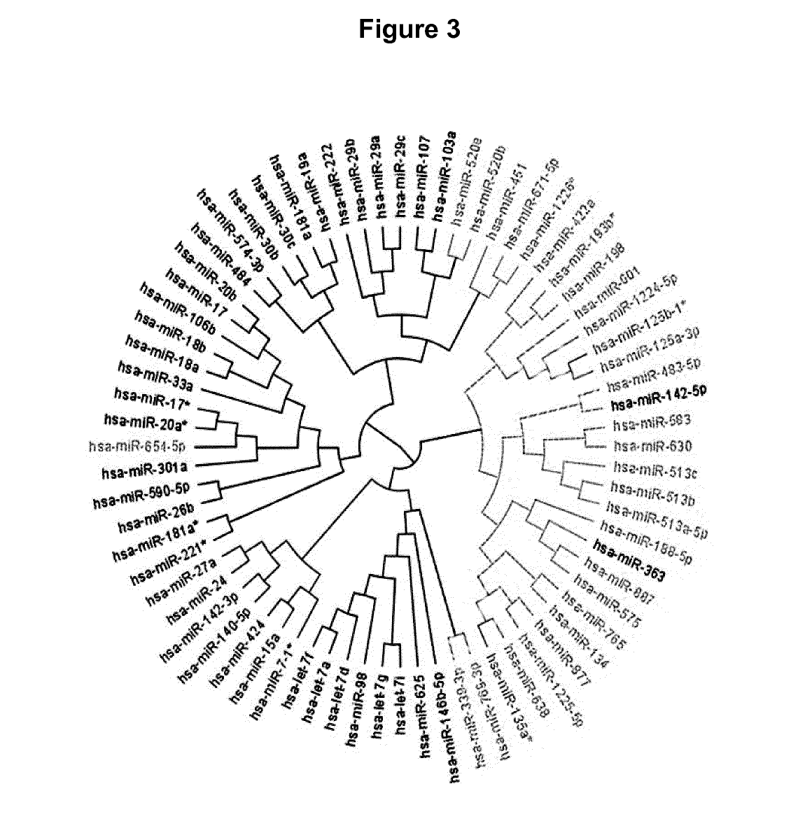

The results obtained in the multiple alignment analysis are represented by a cladogram (FIG. 3). Obtaining this cladogram was performed using Geneious software. Through an unbiased search to identify sequence motifs that were highly represented in the miRNAs, the Cosmo bioconductor kit was used [16]. The ZOOPS model was used to seek sequence motifs of 4-8 nucleotides in length that were highly represented in the miRNAs, using as background those sequences of the miRNAs motifs that showed no differential expression between cells and EVs. A Markov model of order 0 was assumed for the background sequences. All motifs showed an E-value<10⁻⁴.

The cladogram grouped the mature miRNA according to their tendency to be located within cells or within the EVs (FIG. 3). Moreover, paralogous miRNA which have similar sequences but differ in their chromosomal location (and hence in their levels of transcription) always show the same tendency to be located either in EVs or in cells. By contrast, mature miRNA complementary strands, derived from the same pre-miRNA and therefore are expressed at similar levels, but have different sequences, may differ in their tendency to be located inside the cells compared to inside the EVs. Therefore, the displayed data show that the mature miRNA sequence is important in determining its classification in EVs.

An unbiased search for the sequence motifs that were highly represented in the EVmiRNAs or CLmiRNAs, revealed the existence of two sequence motifs, significantly represented in the EVmiRNA sequences, preferably at the 3′region of the regulatory nucleic acid, which were called EV motifs and in turn, showed three sequence motifs significantly represented in the CLmiRNAs sequences, which were named CLmotifs. The EVmotifs identified in the present invention are: GGAG and N₁CCN₂, wherein N₁ C or U and N₂: U or G and the CLmotifs are AN₃CAUN₁. AGGUAGUA and N₁UGCACUN₄; wherein N₁: C or U; N₃: C or A and N₄: G or A.

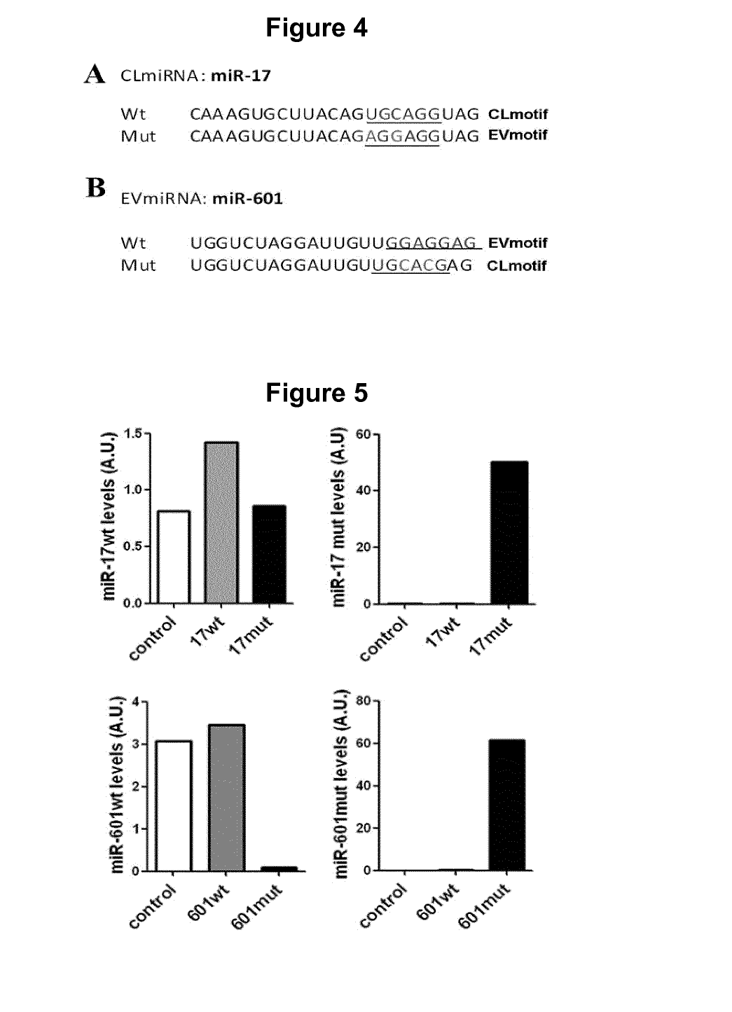

To determine whether such sequence motifs, EV motifs and CL motifs represented significantly in the CLmiRNAs or EVmiRNAs, respectively, are involved in packaging the RNAs into the EVs or their sequestration inside the cells, the CLmiRNA motifs of miR-17 and EVmiRNA of miR-601 were cloned in retroviral vectors in order to subsequently, via directed mutagenesis, transform the CLmotif of miR-17 into an EVmotif, giving rise to a mutated miR-17 (miR-17mut) (FIG. 4A) and, conversely, transforming EVmotif of miR-601 into a CLmotif, resulting in a mutated miR-601 (miR-601 mut) (FIG. 4B).

For this purpose, the Jurkat cell line was transduced by retroviral infection with plasmids expressing miR-17mut or miR-601mut. Briefly, for the cloning of CLmiRNA motifs of miR-17 (UGCAGG) for the retroviral vector, primers used were SEQ ID NO: 1 and SEQ ID NO: 2 and for the cloning of EVmiRNAs motifs of miR-601 (GGAGGAG), primers used were SEQ ID NO: 3 and SEQ ID NO: 4, respectively. These primers were cloned in the vector pGEM-T (Promega) and subsequently in pLVX-AcGFP1-C1, for miR-17 or pMSCV-GFP for miR-601. Directed mutagenesis was performed with the QuickChange Site-Directed mutagenesis kit (Stratagene). The constructions obtained were verified by sequencing their DNA. To obtain the Jurkat cells expressing the miRNA-17mut or miRNA-601mut. HEK293T cells were used, which were co-transfected (Lipofectamine2000; Invitrogen) with the plasmids obtained previously encoding miR-17mut or miR-601mut and the helper plasmids pCMV-ΔR8.91 and pMD2.G-VSV-G in the case of miR-601mut or pCLAmpho helper plasmid in the case of miR-17mut. After 48-72 h the supernatants were collected, filtered (0.45 μm) and added to cultures of Jurkat cells. Subsequently, the transduced cells were centrifuged (1200 g, 2 h), incubated for 4 hours at 37° C.; they were selected with puromycin (4 μg/ml), thereby obtaining cells overexpressing the mutated specific miRNAs.

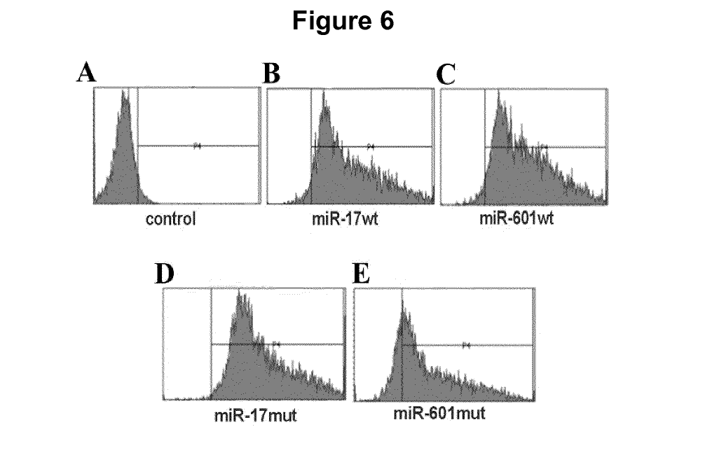

The differential expression of the wild-type miRNAs or control versus mutated miRNA was analyzed using RT-PCR with specific LNA type primers, which allow to discriminate between wild-type sequences and mutated sequences of miRNAs (FIGS. 5 and 6). Briefly, for gene expression analysis of mutated and wild-type miRNAs, the RT-PCR technique was used. Based on the RNA isolated from cells expressing mutated or wild-type miRNAs, cDNA was synthesized using conventional techniques. Mature miRNAs were quantified using the miRCURY LNA Universal RT microRNA PCR kit (Exiqon) using LNA primers for microRNAs (Exiqon) and SybrGreen PCR Master Mix (Applied Biosystems). PCR reactions were performed in triplicate. Data from the quantitative expression of the miRNAs were acquired and analyzed using the ABI Prism 7900HT Sequence Detection System (Applied Biosystems). RNU1A1 and RNU5G genes were used as endogenous controls and. In absolute quantifications, synthetic nucleotides (Exiqon) were serially diluted 1/10 at known concentrations and then transcribed and amplified, calculating the absolute concentration of each miRNA from the standard curve. To normalize the samples 10⁸ copies of synthetic RNA spike-in (Exiqon) was added to each one. The ratio EV/CL was calculated by directly dividing the number of copies of the miRNAs in EVs between the number of copies of miRNAs in cells.

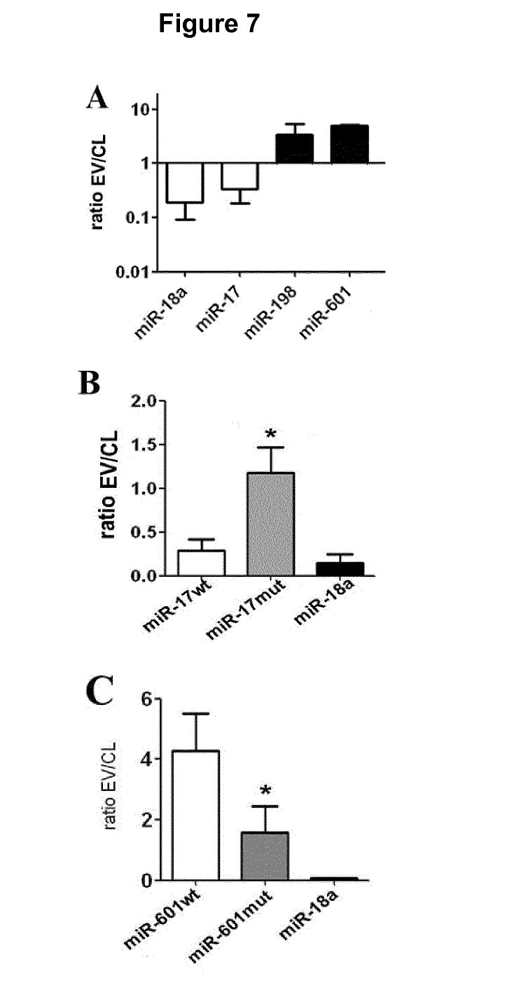

Calculation of the number of copies of each of the miRNAs, and the ratio EV/CL of each miRNA, revealed that the EVmiRNAs motifs of miR-198 and miR-601 showed a higher ratio EV/CL that CLmiRNAs motifs of miR-17 and miR-18a (FIG. 7A), in agreement with the data obtained from the microarray. However, when the CLmotif of miR-17 is converted by directed mutagenesis in an EVmotif (GGAG) (miR-17mut), the ratio EV/CL increases (FIG. 7B). Furthermore, when the EVmotif (GGAG) of miR-601 was transformed into a CLmotif (miR-601mut), the ratio EV/CL of miR-601 decreased (FIG. 7C), indicating that the sequence motif GGAG is responsible for directing the miRNAs packaging into in EVs.

### Example 3

### HnRNP Proteins Specifically Bind to EVmiRNAs

A protein from the hnRNP family, specifically hnRNPA2B1 protein, is an ubiquitous protein that regulates trafficking of mRNAs to the axons of nerve cells [17].

To analyze the molecular mechanism that controls the packaging of miRNAs presenting the motifs of the invention in EVs, a search was performed of the proteins present in EVs and which bind specifically to EVmiRNAs. For this purpose, extracts of isolated EVs from human T lymphoblasts were incubated in the presence of streptavidin beads coated with biotinylated EVmiRNA miR-198, or with biotinylated CLmiRNA miR-17. The analysis of EVs-coupled beads was performed by FACS. Briefly, Evs were obtained by ultracentrifugation and sucrose gradient flotation, resuspended in PBS and coupled to 4 μm aldehyde-sulfate beads (Invitrogen) overnight at room temperature in rotation. Beads were washed and blocked for 60 min at room temperature in 4% BSA in PBS. For intracellular staining, bead-bound Evs were permeabilized and fixed for 5 min at room temperature with 0.2% TX, 2% FA in PBS. Beads were incubated with antihnRNPA2B1 (Santa Cruz) or anti-CD81 (5A6, Santa Cruz) for 1 h at 4° C., washed and incubated with Alexa-488-goat-anti-mouse IgG (Invitrogen) for 30 min. Beads were acquired on a FACSCalibur (BD) and data were analyzed with FlowJo software (Tree Star). Negative controls were obtained with EV-coupled beads incubated with the secondary antibody. The proteins collected were identified by high performance mass spectrometry. As negative controls, uncoated or poly-A-coated streptavidin beads were used. Briefly, proteins were in-gel digested using a previously-described protocol [18]. Briefly, 75 μl of beads were suspended in 25 μl sample buffer and loaded in 2.8-cm-wide wells of an SDS-PAGE gel. The run was stopped as soon as the front entered 3 mm into the resolving gel; the protein band was visualized by Coomassie staining, excised and digested overnight at 37° C. with 60 ng/μl trypsin at 5:1 protein:trypsin (w/w) ratio in 50 mM ammonium bicarbonate, pH 8.8 containing 10% ACN. The resulting tryptic peptides were extracted by 1 h incubation in 12 mM ammonium bicarbonate, pH 8.8. TFA was added to a final concentration of 1% and the peptides were finally desalted onto C18 Oasis cartridges and dried down. Peptide identification by mass spectrometry was performed as described [19]. Briefly, the peptides were resuspended and injected onto a C-18 reversed phase (RP) nano-column (75 μm 2 I.D.×25 cm, Acclaim PepMap100, Thermo Scientific) and analyzed in a continuous acetonitrile gradient consisting of 0-43% B in 90 min, 50-90% B in 1 min (B=90% acetonitrile, 0.5% acetic acid). A flow rate of ca. 300 nL/min was used to elute peptides from the RP nano-column to an emitter nanospray needle for real time ionization and peptide fragmentation on an LTQ XP Orbitrap mass spectrometer (Thermo Fisher, San José, Calif., USA). An enhanced FT spectrum (30.000 resolution) followed by MS/MS spectra from most intense ten parent ions were performed along the chromatographic run (130 min). Dynamic exclusion was set at 30 s. The MS/MS raw files were searched against the Human Swissprot database (Uniprot release 14.0, 19929 sequence entries for human) supplemented with porcine trypsin and bacterial Streptavidin. SEQUEST results were validated using the probability ratio method [20] and false discovery rates calculated using the refined method [21]. Peptide and scan counting was performed assuming as positive events those with a FDR equal to or lower than 5%.

The functional analysis of the precipitated proteins revealed a predominant association related to biological functions directed to post-transcriptional modifications of RNA (Table 1).

TABLE 1
Number of peptides from each of the proteins listed in
the table, which were identified by mass spectrometry.

| | | | poly-A | Beads |
|---|---|---|---|---|
| | CLmiRNA | EVmiRNA | (control) | (control) |
| hnRNPA2B1 | 0 | 4 | 0 | 0 |
| hnRNPA1 | 0 | 2 | 0 | 0 |
| HNRPC | 0 | 1 | 0 | 0 |
| HNRPU | 1 | 3 | 0 | 0 |
| RS4X | 0 | 3 | 0 | 0 |
| RS3 | 1 | 3 | 0 | 0 |
| NUCL | 6 | 9 | 8 | 0 |

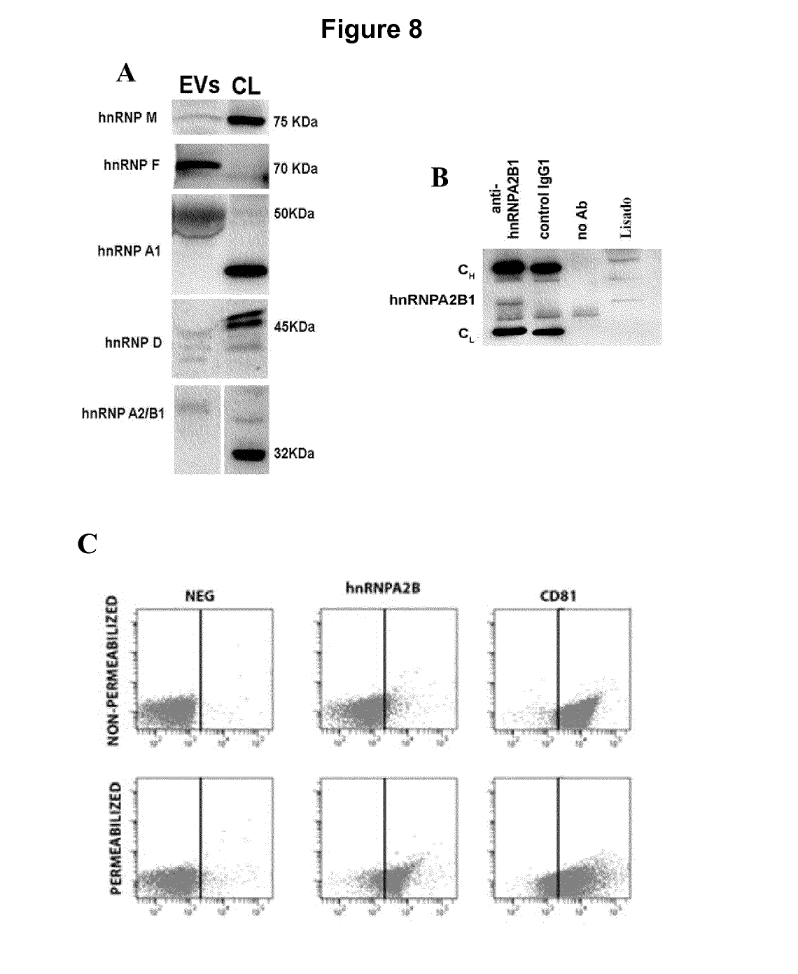

As seen in Table 1, the precipitated proteins included several proteins from the hnRNP family and furthermore, the presence of some of them inside EVs was confirmed by Western blot analysis, as seen in FIG. 8. To perform the Western blot, cells and EVs were lysed in lysis buffer (25 mM Tris pH 8, 150 mM NaCl, 2 mM MgCl2, 0.5% NP-40) with a cocktail of protease inhibitors (Complete, Roche). Proteins were separated on acrylamide/bisacrylamide gels 10% and were transferred to a nitrocellulose membrane. The membranes were incubated with specific primary antibodies (5 mg/ml) compared to the proteins shown in FIG. 8A and with secondary antibodies conjugated with peroxidase (5 mg/ml). Antibodies used were: mouse anti-hnRNPA1 (Sigma), mouse anti-hnRNPA2B1 (Santa Cruz), rabbit anti-hnRNPF (Abcam), mouse anti-hnRNPM (Abcam), rabbit anti-hnRNPD (Upstate), goat anti-mouse peroxidase (Thermo Scientific) and goat anti-rabbit peroxidase (Thermo Scientific).

FACS analysis showed that hnRNPA2B1 fluorescence is higher in permeabilized EVs than in non-permeabilized EVs (FIG. 8C), indicating that the protein locates inside the EVs. As shown in FIG. 8A and Table 1, two proteins of the hnRNP family, specifically, the hnRNPA2B1 and hnRNPA1 proteins, bind exclusively to EVmiRNA, showing no binding to CLmiRNA or poly-A controls (Table 1). Specific binding of the protein hnRNPA2B1 to miR-198 was verified by immunoprecipitation of ribonucleoprotein hnRNPA2B1 obtained from EV lysates followed by a qPCR analysis of miRNAs expression.

The ribonucleoprotein immunoprecipitation technique was performed by washing in PBS 0.01% of Tween Dynabeads Protein G (50 μl) (Invitrogen). Dynabeads were then resuspended in PBS/Tween buffer 0.01% containing 10 μg of anti-mouse hnRNPA2B1 (Santa Cruz) or a control antibody anti-mouse IgG (Santa Cruz) and were incubated overnight at 4° C. EVs were isolated as previously described, then resuspended in cold PBS and subjected to favour binding to a beam of UV light (120 mJ/cm² at 254 nm) (Stratagene UV crosslinker, Stratagene).

EVs were then ultracentrifuged at 100,000 g for 1 h, resuspended in lysis buffer (25 mM Tris pH=8, 150 mM NaCl, 2 mM MgCl₂, 0.5% NP-40, 5 mM DTT protease inhibitors and 40 U/ml RNase inhibitor (Invitrogen)) and incubated for 1 h at 4° C. with pre-washed Dynabeads prior to rinsing. Rinsed lysates were incubated with Dynabeads conjugated with antibody for 1.5 h at 4° C. Subsequently, the Dynabeads were washed twice with the aforementioned lysis buffer and a further three times with lysis buffer containing 900 mm NaCl and 1% NP-40, and once more with a standard lysis buffer. Then, the Dynabeads were transferred to clean tubes and washed with lysis buffer (0.05% NP-40).

For Western blot analysis, the specific loading buffer of the proteins for Western blot (Fermentas) was added to the Dynabeads, and then the samples were boiled at 70° C. for 10 min and subsequently processed for immunoblotting and to display hnRNPA2B1 protein expression.

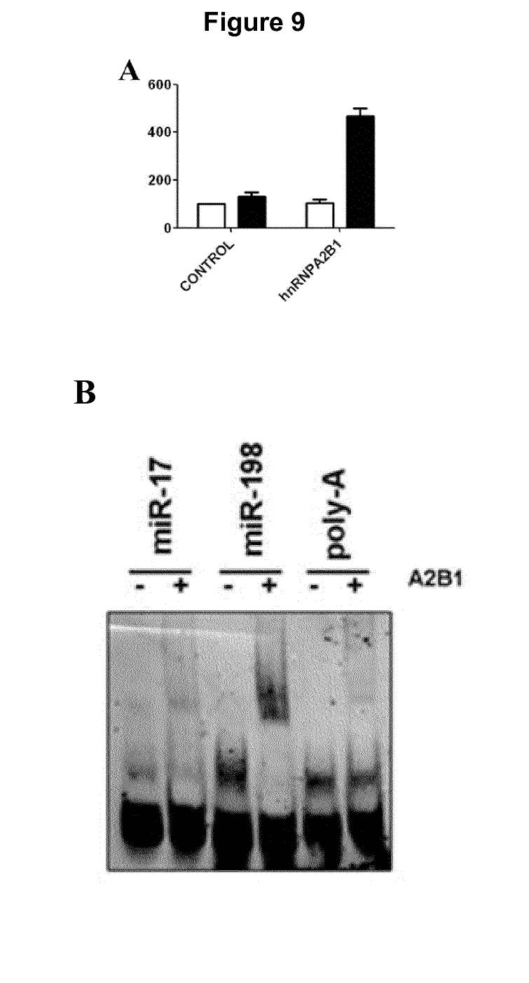

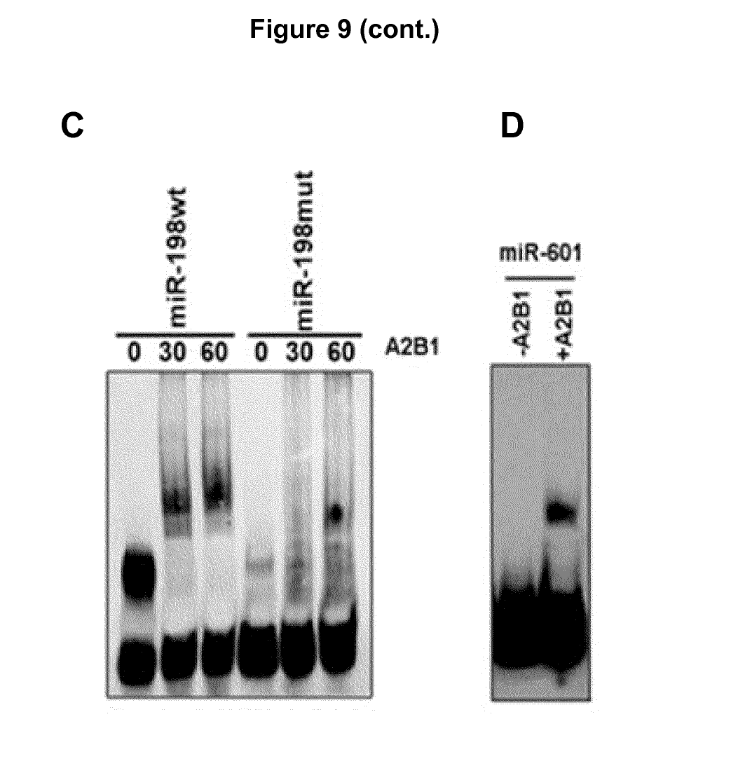

Furthermore, the expression analysis by qPCR showed that the hnRNPA2B1 protein immunoprecipitates were able to amplify miR-198, but not the miR-17, demonstrating specific binding of the protein hnRNPA2B1 and miR-198 in EVs in vivo (FIG. 9A). In order to obtain expression results by qPCT, 700 μl of Qiazol lysis reagent (Qiagen) was added to the Dynabeads coated with anti-hnRNPA2B1 or anti-IgG1 (control) and the samples were vortexed for 1 min. RNA extraction, reverse transcription and qPCR were performed following standard procedures.

To confirm specific binding of the protein hnRNPA2B1 and miR-198 electrophoretic mobility shift assay (EMSA) was performed (FIG. 9B). Briefly, samples of the miRNAs or of biotinylated poly-A (1 nmol) (Dharmacon) were incubated (when indicated) with 3 μg of purified human protein hnRNPA2B1 (Origene) in an EMSA buffer (10 mM HEPES pH 7.3, 5 mM MgCl₂, 40 mM KCl, 1 mM DTT, 5% glycerol, 5 μg tRNA) for 20 min at room temperature. EMSA assay was performed with the EMSA LightShift chemiluminescence kit (Pierce). Said FIG. 9B shows specific binding of miR-198 to the hnRNPA2B1 protein. This binding was inhibited when the EVmotif of miR-198 was mutated (FIG. 9C), indicating that the binding of hnRNPA2B1 to miR-198 is dependent on the presence of the EVmotif. HnRNPA2B1 also binds other EVmiRNAs such as miR-601 (FIG. 9D).

In order to analyze the role of the protein hnRNPA2B1 in the packaging of miRNAs in EVs, an assessment was carried out on the muting or increased expression effect of said protein hnRNPA2B1 in Jurkat T cells on the expression profile of the miRNA present in the EVs (FIG. 10).

To perform increased expression assays of the protein hnRNPA2B1, Jurkat T cells were transfected by electroporation with a plasmid expressing protein hnRNPA2B1-GFP (Origene). These cells were then resuspended in Opti-MEM (GIBCO, 5×10⁷ cells/ml) with 30 μg of plasmid DNA and electroporated with a Gene Pulser XCell (Bio-Rad) at 1200 pFa, 240 mV for 30 ms in 4 mm Bio-Rad cuvettes (Bio-Rad). HnRNPA2B1 GFP-positive cells were analyzed by flow cytometry on a flow cytometer FACSCanto and FACSDiva software (BD Biosciences). Cells showing increased expression of the protein hnRNPA2B1 were separated from the rest by FACSAria sorter (BD Biosciences).

To perform silencing tests of the hnRNPA2B1 protein, Jurkat T cells were transfected twice at 48-hour intervals between each transfection, with siRNAs groups with regard to the hnRNPA2B1 protein, said siRNAs (Eurogentec) being represented by the sequences SEQ ID NOs. 5-12. Control cells were transfected with a control siRNA represented by SEQ ID NO. 13 and SEQ ID NO: 14 (Eurogentec). The cells were then resuspended in Opti-MEM (GIBCO, 5×10⁷ cells/ml) with 2 μM of siRNA and were electroporated with a Gene Pulser Xcell (Bio-Rad) at 240 mV for 28 ms in 4 mm Bio-Rad cuvettes (Bio-Rad).

The results showed that hnRNPA2B1 protein silencing significantly decreased EVmiRNA levels of miR-198 in EVs, whereas no significant changes were observed in CLmiRNA levels of miR-18a (FIG. 11A); by contrast, overexpression of hnRNPA2B1 protein increased miR-198 levels in EVs (FIG. 11B). These data indicate that the protein hnRNPA2B1 specifically controls EVmiRNAs packaging in the EVs.

### Example 4

### The Loading of miRNAs into EVs is Controlled by hnRNPA2B1 Sumoylation

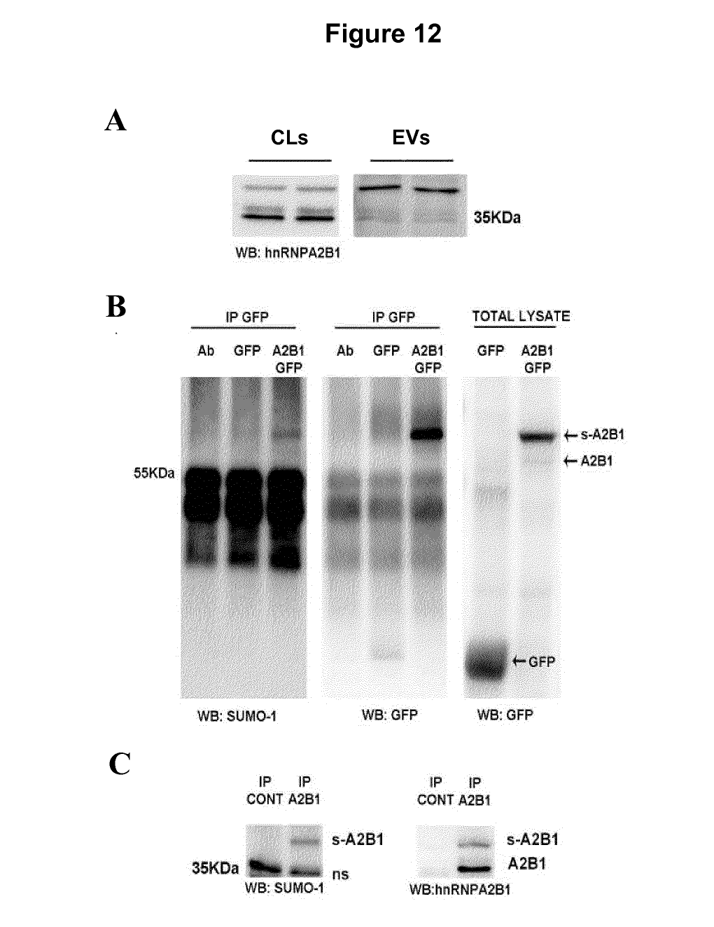

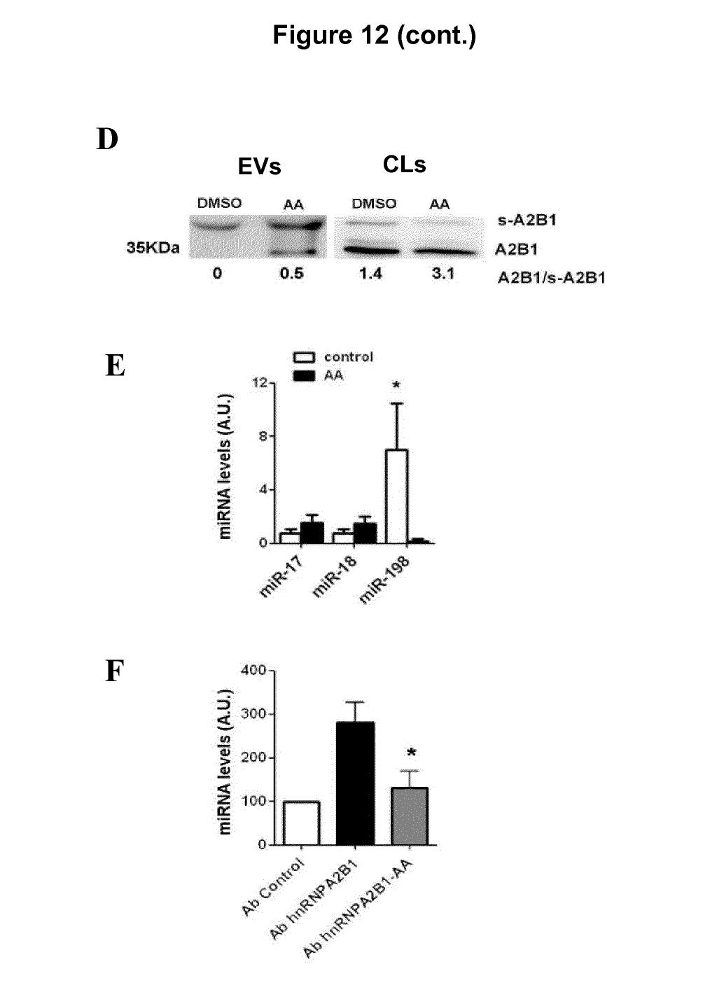

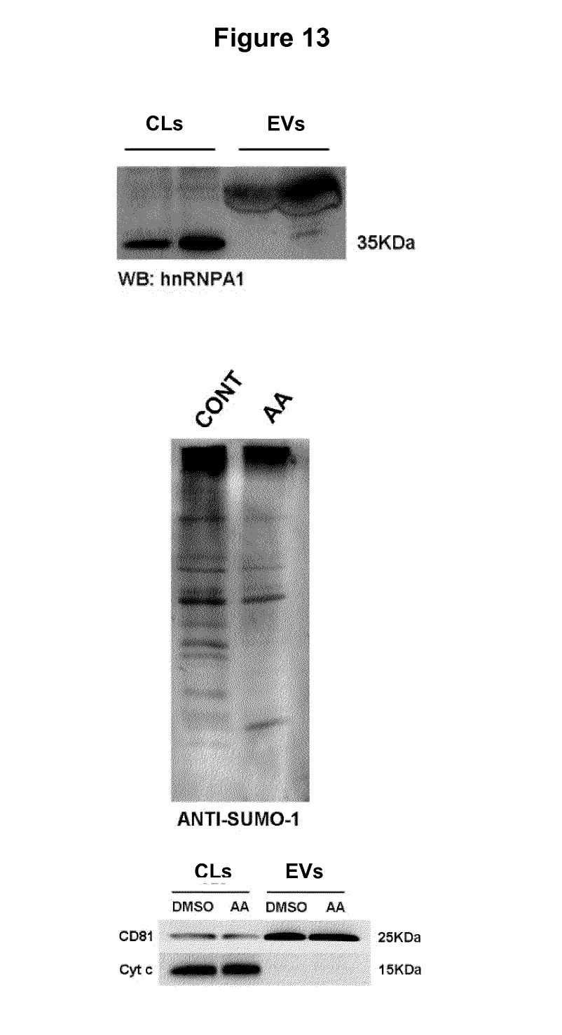

Western blot analysis of hnRNPA2B1 (FIG. 12A) and hnRNPA1 (FIG. 13A) from EVs revealed a higher molecular weight (approximately 10-12 kDa more) in comparison with cells, suggesting that these proteins might be postranslationally modified in EVs. Several hnRNPs including hnRNPA1, are known to attach to small ubiquitin-related modifier (SUMO) [22], and the observed molecular weight changes in EVs are consistent with sumoylation. Moreover, in silico analysis of the hnRNPA2B1 protein sequence identified several predicted sites for SUMO conjugation.

To assess hnRNPA2B1 sumoylation, HEK293T cells were co-transfected with SUMO-1 and hnRNPA2B1-GFP or GFP plasmids. After GFP immunoprecipitation, SUMO-1 was detected in cells transfected with hnRNPA2B1GFP but not in cells transfected with GFP alone (FIG. 12B). Sumoylation of hnRNPA2B1 was also demonstrated in T cells by detection of SUMO-1 after hnRNPA2B1 immunoprecipitation (FIG. 12C).

To confirm whether the higher molecular weight of EVs hnRNPA2B1 was due to sumoylation, we cultured Jurkat T cells in the presence of the specific sumoylation inhibitor anacardic acid (AA) [23]. Briefly, cells were cultured with anacardic acid (100 μM) (Sigma) or 6 DMSO for 4 h at 37° C. Cells were then diluted 1:4 and incubated overnight. EVs and cells were resuspended in buffer 1 for western blotting or hnRNPA2B1 immunoprecipitation (FIG. 13B). Whereas untreated EVs contained barely detectable amounts of low-molecular weight hnRNPA2B1, in the presence of AA the low-molecular weight band could be readily detected (FIG. 12D).

These results indicate that the higher molecular weight of EV hnRNPA2B1 is due to sumoylation, and that sumoylated hnRNPA2B1 is preferably sorted to EVs. Further analysis revealed that treatment with AA reduced the levels of miR-198 in EVs, whereas the levels of miR-17 and miR-18 did not change (FIG. 12E). Moreover, hnRNPA2B1 IP-qPCR experiments showed that there was less miR-198 bound to hnRNPA2B1 when sumoylation was inhibited (FIG. 12F), while EV secretion or cell death did not change (FIG. 13C). These data indicate that hnRNPA2B1-mediated loading of EVmiRNAs into EVs is controlled by the sumoylation of this protein.

The results shown herein demonstrate the existence of short sequence motifs capable of directing regulatory nucleic acids, preferably RNAs into EVs, the mechanism being enhanced by the binding of hnRNP family proteins to such RNAs. More specifically, hnRNPA2B1, which is present in EVs, binds the EVmiRNA directly and controls its loading into these microvesicles. In addition, hnRNPA2B1 in EVs is preferentially sumoylated, and this sumoylation is important for the loading of EVmiRNAs into EVs. So, in this sense, sumoylated hnRNPA2B1 directs the loading of certain miRNAs into EVs through recognition of specific short motifs disclosed in the present invention. These RNA motifs are suitable for the artificial loading of selected small regulatory RNAs into EVs, preferably into exosomes and may prove to be suitable tools for the engineering of EVs for gene therapy.

### REFERENCES

1. C. Thery, M. Ostrowski, E. Segura, Nat Rev Immunol 9, 581 (2009).
2. M. Mittelbrunn, F. Sanchez-Madrid, Nat Rev Mol Cell Biol 13, 328 (2012).
3. H. Valadi et al., Nat Cell Biol 9, 654 (2007).
4. M. Mittelbrunn et al., Nat Commun 2, 282 (2011).
5. A. Montecalvo et al., Blood 119, 756 (2012).
6. E. Hergenreider et al., Nat Cell Biol 14, 249 (2012).
7. C. D'Souza-Schorey, J. W. Clancy, Genes Dev 26, 1287 (2012).
8. H. Peinado et al., Nat Med, (2012).
9. C. Thery et al., Nat Immunol 3, 1156 (2002).
10. C. P. Lai, X. O. Breakefield, Front Physiol 3, 228 (2012).
11. L. Alvarez-Erviti et al., Nat Biotechnol 29, 341 (2011).
12. H. W. Hwang, E. A. Wentzel, J. T. Mendell, Science 315, 97 (2007).
13. G. Wang et al., Cell 142, 456 (2010).
14. G. K. Smyth, Stat Appl Genet Mol Biol 3, Article3 (2004).
15. A. Muniategui et al., PLoS One 7, e30766 (2012).
16. O. Bembom, S et al. Stat Appl Genet Mol Biol 6, Article8 (2007).
17. T. P. Munro et al., J Biol Chem 274, 34389 (1999).
18. Bonzon-Kulichenko, E. et al. Mol Cell Proteomics 10, M110 003335 (2011).
19. Goetz, J. G. et al. Cell 146, 148-163 (2011).
20. Martinez-Bartolome, S. et al. Mol Cell Proteomics 7, 1135-1145 (2008).
21. Navarro, P. & Vazquez, J. J Proteome Res 8, 1792-1796 (2009).
22. Li, T. et al. Proc Natl Acad Sci USA 101, 8551-8556 (2004).
23. Fukuda, I. et al. Chem Biol 16, 133-140 (2009).

1-46. (canceled)

47. A method for introducing nucleic acid into extracellular vesicle, the method comprising:
introducing into a cell a nucleic acid comprising at least one sequence motif selected from GGAG and/or N1CCN2, wherein N1 is C or U and N2 is U or G, such that said nucleic acid is incorporated into said extracellular vesicle, such that said nucleic acid is introduced into extracellular vesicle.

48. The method of claim 47, further comprising collecting said extracellular vesicle.

49. The method of claim 47, wherein said extracellular vesicle is an exosome.

50. The method of claim 47, wherein said sequence motif is GGAG.

51. The method of claim 47, wherein said nucleic acid is an mRNA, a miRNA or a siRNA.

52. The method of claim 47, wherein said nucleic acid is in complex with a heterogenous nuclear ribonucleoprotein (hnRNP).

53. The method of claim 52, wherein said heterogenous nuclear ribonucleoprotein (hnRNP) is hnRNPA2B1 or hnRNPA1.

54. The method of claim 52, wherein said hnRNP is sumoylated.

55. A method for producing an extracellular vesicle comprising a nucleic acid, the method comprising:
providing said nucleic acid;
introducing into said nucleic acid at least one sequence motif selected from GGAG and/or N1CCN2, wherein N1 is C or U and N2 is U or G, thereby generating a nucleic acid capable of being loaded into an extracellular vesicle; and
introducing said nucleic acid capable of being loaded into an extracellular vesicle into a cell, such that an extracellular vesicle comprising a nucleic acid is produced.

56. The method of claim 55, further comprising collecting said extracellular vesicle.

57. The method of claim 55, wherein said extracellular vesicle is an exosome.

58. The method of claim 55, wherein said sequence motif is GGAG.

59. The method of claim 55, wherein said sequence motif is introduced at the 3′ region of said nucleic acid.

60. The method of claim 55, wherein said nucleic acid is an mRNA, a miRNA or a siRNA.

61. The method of claim 55, wherein said nucleic acid is in complex with a heterogenous nuclear ribonucleoprotein (hnRNP).

62. The method of claim 61, wherein said heterogenous nuclear ribonucleoprotein (hnRNP) is hnRNPA2B1 or hnRNPA1.

63. The method of claim 61, wherein said hnRNP is sumoylated.

64. A method for producing a nucleic acid capable of being loaded into an extracellular vesicle, said method comprising introducing into a nucleic acid at least one sequence motif selected from GGAG and/or N1CCN2, wherein N1 is C or U and N2 is U or G, such that said nucleic acid capable of being loaded into an extracellular vesicle is produced.

65. The method of claim 64, wherein said extracellular vesicle is an exosome.

66. The method of claim 64, wherein said sequence motif is GGAG.

67. The method of claim 64, wherein said sequence motif is introduced at the 3′ region of said nucleic acid.

68. The method of claim 64, wherein said nucleic acid is an mRNA, a miRNA or a siRNA.

69. An extracellular vesicle comprising the nucleic acid produced by the method of claim 64.

70. The extracellular vesicle of claim 69, wherein said extracellular vesicle is an exosome.

71. The extracellular vesicle of claim 70, wherein said nucleic acid is in complex with a heterogenous nuclear ribonucleoprotein (hnRNP).

72. The extracellular vesicle of claim 74, wherein said heterogenous nuclear ribonucleoprotein (hnRNP) is hnRNPA2B1 or hnRNPA1.

73. The method of claim 71, wherein said hnRNP is sumoylated.

74. A gene therapy method for treating a subject in need thereof, comprising administering to said subject the extracellular vesicle of claim 69.
